(19) United States
(12) Patent Application Publication
(10) Pub. No.: US 2008/0102097 A1
(43) Pub. Date: May 1, 2008

(54) Device and method for treating osteolysis using a drug depot to deliver an anti-inflammatory agent

(76) Inventor: John M. Zanella, Cordova, TN (US)

Correspondence Address: FOX ROTHSCHILD, LLP, 997 LENOX DRIVE, LAWRENCEVILLE, NJ, 08648, US

(21) Appl. No.: 11/591,243

(22) Filed: Oct. 31, 2006

(51) Int. Cl. A61F 2/02; A61K 31/401; A61K 31/366

(52) U.S. Cl. 424/423; 514/423; 514/460; 514/301

(57) ABSTRACT

The present invention relates to a device and method of treating an osteo-degenerative disease followed by an optional procedure using an osteoconductive material to accelerate healing of bone tissue damaged by the disease. The device for treating the bone degenerative disease comprises an in situ drug depot that time releases at least one anti-inflammatory agent. The method for treating an osteo-degenerative disease comprising: a) the combination of at least one anti-inflammatory agent with a flowable drug carrier, b) delivering the flowable drug carrier/anti-inflammatory mixture to a bone lesion, c) in situ curing of the mixture forming a rigid drug depot, d) wherein the drug depot bio-resorbs releasing the anti-inflammatory agent over time to treat a degenerative bone disease), and e) followed by an optional treatment using an osteoconductive material in combination with a growth factor.

### FIELD OF INVENTION

The present invention relates to a device and method of treating an osteo-degenerative disease (e.g. osteolysis).

### BACKGROUND OF THE INVENTION

Severe joint damage often requires a patient to undergo total joint arthroplasty to relieve pain and restore motion to a damaged joint. Approximately 1,000,000 total hip replacement surgeries are performed annually world wide. However, prosthetic implants wear with time, become loose in the bone cavity, and cause osteo-degenerative diseases. Nearly 30% of implant recipients have their prosthetics removed and replaced within ten to fourteen years after the initial surgery.

Osteolysis is a particular type of bone degeneration caused by the body's natural inflammation responses to wear particles (e.g. debris) emitted by a worn prosthetic. This disease is often a cause for prosthetic loosening. When a prosthesis is in motion, surfaces glide across each other forming debris particles of ultra-high molecular-weight polyethylene (UHMWPE), metal, ceramic, and cement. These particles will migrate to the synovial cavity and the bone-implant interface. Inflammation causes the bone tissue adjacent to the prosthesis to resorb away from the surface of the implant. This action loosens the prosthesis from the bone tissue causing stiffness and pain in the patient.

The large inflammation response is due in part to the debris particles' artificial chemistry. During phagocytosis, macrophages can consume but not digest debris particles from the prosthesis. The inability to digest the debris particles lead to an increased, yet vain, macrophage production of inflammation regulators (cytokines) and growth factors, for example Tumor Necrosis Factor-α (TNF-α), interleukin-1β (IL-1β) and interleukin-6 (IL-6), PDGF, M-CSF, and GM-CSF.

The regulators direct a heavy foreign-body macrophage response toward debris particles in the synovial cavity and the bone-implant interface. Because macrophage cells do not possess enzymes that can readily digest the heavy polymer or metal particles emitted by the prosthesis, the degree of inflammation response is a function of the size, distribution, and concentration of debris particles in and about the bone-implant interface.

Macrophage secretion of pro-inflammatory cytokines in response to phagocytosis of debris particles contribute to osteoclast cell formation about the prosthesis, where osteoclast cells function as a natural part of bone tissue rejuvenation via bone resorption. It follows that if pro-inflammatory cytokines create a high concentration of osteoclast cells, then the natural bone resorption process increases as well. This action leads to significant bone loss that compromises the stability of the prosthesis.

One can block pro-inflammatory cytokine production at the macrophage nucleus during DNA-RNA transcription. In the nucleus, DNA encompassing the target gene expression for a pro-inflammatory cytokine is activated by transcription factors, such as for example, proteins belonging to the NF-κB family). Pro-inflammatory cytokines, such as TNF-α have been shown to directly activate the NF-κB pathway creating an auto-regulatory loop resulting in chronic inflammation and pain.

Blocking NF-κB pathways will inhibit production of pro-inflammatory cytokines and their action mechanisms that promote inflammation. Inhibiting NF-κB pathways, which slows production of pro-inflammatory cytokine by macrophage cells, including the decrease of TNF-α formed osteoclastic cells that promote resorption.

Standard treatment for osteolysis includes treating pain symptoms with analgesics and steroids before subjecting the patient to a revision surgery. Currently, there are no treatments specifically approved for treating osteolysis other than replacing the loose prosthesis.

U.S. Pat. No. 6,884,247 discloses methods for treating osteolytic bone lesions that include making two holes in the bone at the bone-implant interface, applying a negative pressure source to one hole, and injecting a flushing fluid into the other. The method of the '247 patent calls for flushing the fluid line containing a biocompatible material to fill said lesion.

U.S. Pat. No. 6,746,488 discloses a method and apparatus for hindering osteolysis in porous implants by coating said implant with a bioresorbable material, which prevents the infiltration of debris particles into the pores of the implant and allows the ingrowth of new bone tissue into said prosthetic. Neither the '247 nor the '488 patent directly inhibits the root cause of an osteo-degenerative disease such as osteolysis.

Accordingly, there is an immediate need for an improved medical device and method for treating osteo-degenerative diseases like osteolysis.

### SUMMARY OF THE INVENTION

The present invention overcomes the drawbacks of the prior art. The methods and devices of the present invention for treating a bone degenerative disease comprise a drug depot that encapsulates an anti-inflammatory agent, wherein the depot solidifies in situ when placed in a subject.

One aspect of the invention may include, at least one secondary additive capable of treating an osteo-degenerative disease in or near a bone lesion.

Another aspect of the present invention provides a method of treating an osteo-degenerative disease, comprising: a flowable drug carrier mixed with an anti-inflammatory agent and/or secondary additives; comprising delivering the mixture to the diseased bone tissue, curing the mixture in situ via forming a rigid drug depot that encapsulates and time releases the anti-inflammatory, and healing the diseased bone tissue by either time released secondary additives in the mixture and/or a follow-up medical procedure involving a osteoconductive biomaterial with or without a osteoinductive factor. In some aspects of the present invention, the anti-inflammatory agent is a NF-κB inhibitor, capable of blocking the production of pro-inflammatory cytokines at the macrophage nucleus.

The present invention would produce a cascading effect that slows osteo-degenerative diseases such as osteolysis, thereby reducing inflammation and pain while ensuring the integrity of a prosthesis without surgical removal and replacement.

It is an object of the invention where the osteo-degenerative disease is osteolysis brought on by the production and inflammation mechanisms of pro-inflammatory cytokines.

It is another object of the invention wherein the bone lesion is caused by a heavy inflammation response to debris particles emitted by a worn prosthetic implant.

It is an object of the invention where the bone lesion can be adjacent to and/or is in contact with a prosthetic implant.

It is yet another object of the invention wherein the drug depot time releases the at least one anti-inflammatory agent over a period of five months to one year from delivery and in situ curing of a flowable mixture in a bone lesion.

It is an object of the invention wherein the drug depot is formed by in situ curing of a flowable mixture comprising a flowable drug carrier and at least one anti-inflammatory agent that is delivered to a bone lesion.

Another object of the invention includes a flowable drug carrier made from a bioresorbable polymer selected from the group consisting of oligomers, polymers, or combinations thereof of lactic acid, glycolic acid, lactide-co-glycolides, anhydrides, orthoesters, caprolactone, and tyrosin-polycarbonate. In some embodiments of the invention the bioresorbable polymer is selected from the group consisting of elastomers, hydrogels, rigid polymers or combinations thereof.

In yet another object of the invention the flowable drug carrier is a combination of a bioresorbable polymer and a osteoconductive biomaterial. In this object of the invention the biomaterial includes for example bone putty or a ceramic, wherein the ceramic may include for example calcium phosphate, hydroxyapatite, calcium sulfate, bioactive glass or any combination thereof.

Yet another object of the invention includes the at least one anti-inflammatory being an NF-κB inhibitor capable of blocking the production and inflammation pathways of a pro-inflammatory.

Another object of the invention includes a flowable mixture further comprising at least one secondary additive.

Yet another object of the invention includes a device wherein the at least one secondary additive is selected from the group consisting of growth factors, antibiotics, analgesics, radiocontrast agents or any combination thereof.

In situ curing involves activation by applying energy to the flowable mixture after delivery to a bone lesion.

Numerous methods are known in the art that are directed to energy for in situ curing. These are for example, light energy, heat energy, radiation energy, electrical energy, mechanical energy, and combinations thereof.

Yet another object of the invention includes in situ curing of the flowable mixture forming a drug depot that encapsulates and time releases at least one anti-inflammatory agent and, optionally, any secondary additives.

It is an object of the invention wherein a delivery device is used to deliver the flowable mixture to a bone lesion.

Yet another object of the invention includes the delivery device being selected from a group comprising a syringe, needle, cannula, or catheter.

Still another object of the invention includes a delivery device having a channel with a cross section not larger than 8 G.

It is an object of the invention wherein the flowable mixture may optionally have at least one biologically active agent.

It is an object of the invention wherein the growth factor is BMP-2 or LMP-1 or combinations thereof.

It is an object of the invention wherein the at least one secondary additive is selected from the group consisting of crystals or powders of salts, calcium carbonate or sodium bicarbonate.

It is yet another object of the invention to disclose a method of treating an osteo-degenerative disease comprising the steps of: a) the combination of an effective amount of at least one anti-inflammatory agent, and, optionally, an effective amount of at least one secondary additive, with an effective amount of a flowable drug carrier forming a flowable mixture; b) delivering the flowable mixture to a bone lesion; c) in situ curing of the mixture forming a rigid drug depot that encapsulates the anti-inflammatory agent; d) wherein the drug depot bio-resorbs thereby time releasing the anti-inflammatory agent to treat a cause of a degenerative bone disease; and d) followed by an optional treatment using an osteoinductive implant.

It is an object of the invention that includes having a method as disclosed wherein the osteo-degenerative disease is osteolysis caused by an inflammation response to debris particles emitted from a prosthetic implant.

It is another object of the invention to disclose a method wherein the energy to activate in situ curing of the flowable mixture is applied during or after delivery of the flowable mixture to a bone lesion.

It is an object of the invention to disclose a method wherein an osteo-inductive material comprises a medical grade purified collagen, a biphasic calcium phosphate (BCP), ceramic granules, and growth factors.

### DETAILED DESCRIPTION OF THE INVENTION

### Definitions

“Flowable mixture,” and “mixture,” refers to a mixture containing a flowable drug carrier, at least one anti-inflammatory agent, and in some embodiments of the present invention, at least one secondary additive, wherein the mixture is in a flowable liquid like state.

“Deliver(s)” used herein generally refers to process of the injecting the flowable mixture into or near a bone lesion.

“NF-κB inhibitor(s)” refer(s) to proteins or small molecules that block the transcription actions of the protein Nuclear Factor-kappaB.

“Bone lesion(s)” refer(s) to any sort of bone tissue damage, injury, hurt, or wound caused by trauma to the tissue or by any sort of osteo-degenerative disease (e.g. osteolysis).

“Drug depot” and “device” refers to a rigid polymer formed after in situ curing of a mixture containing a flowable drug carrier, at least one anti-inflammatory, and, optionally, at least one secondary additive. The rigid polymer encapsulates and releases, by bio-resorption, any anti-inflammatory agents and any optional secondary additives to treat osteolysis and to heal damaged bone tissue caused by the disease.

“Osteolytic bone lesion(s)” or “osteolytic lesion(s)” refers to bone tissue damage, injury, hurt, or wounds caused by osteolysis.

A device and method for treating an osteo-degenerative disease is generally described herein. Although the present invention is primarily intended to treat osteolysis, these descriptions should not be treated as a limitation on the scope of the invention but, rather, any use of specific language and references are for detailing different embodiments of the same. One of ordinary skill in art will appreciate that the present invention can be used in other clinical situations where causes other than osteolysis is the source of damaged bone tissue.

Flowable Drug Carrier and Combinations Thereof

In one embodiment of the invention, the flowable drug carrier can be a bioresorbable polymer or a bioresorbable/non-bioresorbable polymer combination. Examples of bioresorbable polymers for the present invention may include, without limitation: poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof.

A person of ordinary skill in the art will appreciate that a variety of different non-bioresorbable polymers can be combined with the flowable drug carrier to increase the stability of the drug depot when releasing the anti-inflammatory agent. A flowable drug carrier that is a combination of a bioresorbable and a non-bioresorbable polymer will create a drug depot after in situ curing that can provide a solid scaffolding for the surrounding bone tissue as a portion of the drug depot bio-resorbs.

In yet another embodiment, the flowable drug carrier may be physically mixed with biomaterials as secondary additives to the mixture before or during delivery of the mixture to a bone lesion. Suitable biomaterials include, without limitation, different polymers, metals or ceramics. Non-limiting examples of ceramics include calcium phosphate, hydroxyapatite, calcium sulfate, bioactive glass or a combination thereof.

Anti-Inflammatory Agent

Anti-inflammatory agents that act as NF-κB inhibitors are ideally suited for use in the present invention. NF-κB inhibitors block the production and inflammation pathways of proinflammatory cytokines that promote inflammation, osteoclast formation, and osteolysis.

Suitable NF-κB inhibitors may be selected from the group consisting of sulfasalazine, sulindac, clonidine, helenalin, wedelolactone, pyrollidinedithiocarbamate (PDTC), Inhibitor-Kappa B Kinase-β VI, Inhibitor Kappa Kinase III (BMS-345541) and combinations thereof.

Secondary Additives

Secondary additives comprising anti-inflammatory compounds and biologically active agents may be physically mixed with the flowable drug carrier before or during delivery of the mixture to a bone lesion. Suitable examples of biologically active agents may include, without limitation: other anti-inflammatories, growth factors, antibiotics, analgesics and radiocontrast agents.

Other Anti-inflammatories as Secondary Additives

Suitable anti-inflammatories may be added to reduce inflammation that may arise with the introduction of the mixture into a bone lesion. Anti-inflammatory compounds include both steroidal and non-steroidal structures.

Suitable examples of steroidal anti-inflammatory compounds are, without limitation: corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene(fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting examples of non-steroidal anti-inflammatory compounds may include without limitation: nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The variety of compounds encompassed by this group are well-known to those skilled in the art. For detailed disclosure of the chemical structure, synthesis, side effects, etc. of non-steroidal anti-inflammatory compounds, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, New York (1974), each incorporated herein by reference.

Mixtures of these non-steroidal anti-inflammatory compounds may also be employed, as well as the pharmacologically acceptable salts and esters of these compounds.

In addition, so-called “natural” anti-inflammatory compounds are useful in methods of the disclosed invention. Such compounds may suitably be obtained as an extract by suitable physical and/or chemical isolation from natural sources (e.g., plants, fungi, by-products of microorganisms). Suitable examples of such compounds may include, without limitation: candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, sea whip extract, compounds of the Licorice (the plant genus/species Glycyrrhiza glabra) family, including glycyrrhetic acid, glycyrrhizic acid, and derivatives thereof (e.g., salts and esters).

Suitable salts of the foregoing compounds may include, without limitation, metal and ammonium salts. Suitable esters may include, without limitation: C2-C24 saturated or unsaturated esters of the acids, preferably C10-C24, more preferably C16-C24. Specific examples of the foregoing may include, without limitation: oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

Growth Factors as Secondary Additives

Suitable growth factors may be added to the flowable mixture as a secondary additive. Growth factors may be selected from a group consisting of, without limitation: BMP-1, BMP-2, rhBMP-2, BMP-3, BMP-4, rhBMP-4, BMP-5, BMP-6, rhBMP-6, BMP-7 [OP-1], rhBMP-7, BMP-8, BMP-9, BMP-10, BMP-11, BMP-12, BMP-13, BMP-14, BMP-15, BMP-16, BMP-17, BMP-18, Growth and Differentiation Factors, GDF-5, Cartilage Derived Morphogenic Proteins, LIM mineralization protein, platelet derived growth factor (PDGF), transforming growth factor α (TGF-α), insulin-related growth factor-I (IGF-I), insulin-related growth factor-II (IGF-II), fibroblast growth factor (FGF), beta-2-microglobulin (BDGF II), rhGDF-5 and any combination thereof.

Antibiotics as Secondary Additives

Suitable antibiotics may be added to the flowable mixture as a secondary additive. Antibiotics may be selected from the group consisting of, without limitation: nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Analgesics as Secondary Additives

Suitable analgesics maybe added to the flowable mixture as a secondary additive. Analgesics may be selected from the group consisting of, without limitation: opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

Radiocontrast Agents as Secondary Additives

Suitable radiocontrast agents may be added to the flowable mixture as a secondary additive. Radiocontrast agents aid the physician in tracking the delivery of the flowable mixture to the bone lesion. Radiocontrast agents may be selected from a group consisting of, without limitiation: barium and iodine compounds, metal ions, nitroxides, and gadolinium complexes, such as gadodiamine.

Mixing, Delivering, and in Situ Curing of the Flowable Mixture

The flowable mixture may be delivered in or near a bone lesion with a syringe, a cannula connected to a reservoir containing the flowable mixture, where the reservoir can be a syringe and a pump, or a catheter. The channel of the syringe or cannula has a cross section no larger than 8 G. The channel of the catheter has a cross section no larger than 8 G.

After delivery, the flowable mixture maybe cured in situ by the application of energy. The energy source is not important for the instant invention: it can be light energy, heat energy, radiation energy, electrical energy, mechanical energy, or any combination thereof.

Other means of bringing about in situ curing of the flowable mixture includes adding an effective amount of differing curing agents to the mixture before or during delivery of the mixture to a bone lesion. Examples of other curing agents include, but are not limited to, monomers, oligomers, polymers, or combinations thereof of any members of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and/or any combinations thereof.

For example, one embodiment the invention comprises a flowable drug carrier having oligomers of bioresorbable polymers, such as lactic acid and/or glycolic acid and/or anhydrides thereof, and monomers and oligomers of a curable material such as silicone and/or polyurethane. In such an embodiment, the cured solid substance will comprise intermixed units of lactic acid, glycolic acid, polyurethane and silicone.

Curing creates a scaffolding network of “linked” bio-resorbable polymers (i.e. the drug depot) that encapsulates anti-inflammatory agents. The in situ curing process ensures dimensional stability of the bioresorbable polymer during resorption and uniformed delivery of the anti-inflammatory agent in and/or about the bone lesion.

Upon activation, the implant may undergo progressive polymerization with increasing viscosity and, most likely, heat release due to an exothermic reaction. In different embodiments of the invention, the peak temperature of the polymerization is not higher than 75° C., preferably not higher than 60° C., preferably not higher than 50° C. per volume of the administered composition.

The adsorption rate of the drug depot is controlled by many factors such as the chemical bonds within the polymer, the use of solvents, and the bulk flow around the implant. Some polymers are designed to erode via “bulk erosion”; whereas, others are designed to erode via “surface erosion”. Ideally, the drug depot will time release the at least one anti-inflammatory agent and, optionally, any secondary additives over a period of or near 1 year (e.g. 6 months) from in situ curing of the mixture. It is also preferred that in situ curing of the flowable mixture should occur in less than one minute from the start of application, more preferably be finished in less than five minutes, most preferably be finished in less than three minutes.

The drug depot should have an elastic modulus of at least about 100 MPa, wherein at least about 50% of the drug depot bio-resorbs to release the at least one anti-inflammatory agent(e.g. an NF-κB inhibitor) at a rate specific to blocking the production and inflammation pathways of proinflammatory cytokine.

Osteoinductive Material Procedure

Another embodiment of the invention may include an optional procedure, whereby an osteoinductive material is implanted into a bone lesion in a follow-up procedure to the drug depot therapy of the present invention. Using an osteoconductive material laced with growth factors as a follow up procedure will accelerate healing of the bone lesion.

It is well known in the art that a malleable osteoconductive material carrying growth factors can be used to fix bone lesions. An osteoconductive material allows the user to have a malleable drug depot that localizes biological components and allows a bone graft to be shaped based on the surgical environment and patient anatomy.

One such material is disclosed in a co-pending application U.S. patent application Ser. No. 11/497,837 and is herein incorporated by reference. The referenced bone putty is a combination of medical grade purified collagen and a biphasic calcium phosphate (BCP), and ceramic granules, which incorporates osteoinductive factors. The use of this particular putty is a non-limiting example and does not serve as a limitation on the use of differing osteoconductive material as matrix materials for this invention.

Another non-limiting example of a bone putty is detailed in U.S. Pat. No. 6,576,249 and is herein incorporated by reference. The bone putty disclosed in the '249 patent has a useful bulk viscosity and optimum bioabsorbability, where an osteoinductive factor and an NF-κB inhibitor would be admixed with the putty before the implant is delivered to the bone lesion.

Dosage:

Although 0.05 mg of a growth factor (BMP, for example)/g of osteoconductive material, for example purified collagen and a biphasic calcium phosphate (BCP), is an amount sufficient to heal bone defects, the dose of growth factor required to effect osteo-induction is generally more. Accordingly about 0.1 mg to about 3 mg BMP, for example/g of osteoconductive carrier is a preferred range. One example embodiment of the present invention comprises between about 2 mg and about 3 mg per gram (/g), e.g., about 2.5 mg protein/g of a osteoconductive material.

Surgeon Kit:

A surgeon kit is provided for treating an osteo-degenerative disease followed by an optional procedure using an osteoinductive material. The kit is comprised of a container comprising a flowable drug carrier encapsulating an anti-inflammatory agent, optionally, encapsulating secondary additives and/or curing agents; a delivery device; and a curing device. The kit may also include written instructions that indicate a method for treating an osteo-degenerative disease.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A device for treating a bone degenerative disease comprising:
an in situ drug depot that encapsulates and time releases at least one anti-inflammatory agent capable of treating an osteo-degenerative disease in or near a bone lesion.

2. The device of claim 1 wherein the osteo-degenerative disease is osteolysis brought on by the production of inflammation mechanisms.

3. The device of claim 1 wherein the bone lesion is caused by a heavy inflammation response to debris particles emitted by a worn prosthetic implant.

4. The device of claim 1 wherein the bone lesion can be adjacent to and/or is in contact with a prosthetic implant.

5. The device of claim 1 wherein the drug depot time releases the at least one anti-inflammatory agent over a period of five months to one year from delivery of a flowable mixture to a bone lesion.

6. The device of claim 1 wherein the drug depot is formed by in situ curing of a flowable mixture comprising a flowable drug carrier and at least one anti-inflammatory agent that is delivered to a bone lesion.

7. The device of claim 6 wherein the flowable drug carrier is made of a bioresorbable polymer selected from the group consisting of oligomers, polymers, or combinations thereof of lactic acid, glycolic acid, lactide-co-glycolides, anhydrides, orthoesters, caprolactone, and tyrosin-polycarbonate.

8. The device of claim 6 wherein the at least one anti-inflammatory is an NF-κB inhibitor capable of blocking the production and inflammation pathways of TNF-α proteins selected from the group consisting of sulfasalazine, sulindac, clonidine, helenalin, wedelolactone, pyrollidinedithiocarbamate (PDTC), Inhibitor-Kappa B Kinase-β VI, Inhibitor Kappa Kinase III (BMS-345541) or any combination thereof.

9. The device of claim 1 further comprising at least one secondary additive.

10. The device of claim 9 wherein the at least one secondary additive is selected from the group consisting of growth factors, antibiotics, analgesics, radiocontrast agents or any combination thereof.

11. The device of claim 6 wherein in situ curing is activated by applying energy to the flowable mixture after delivery to a bone lesion.

12. The device of claim 11 wherein the energy is selected from the group consisting of light energy, heat energy, radiation energy, electrical energy, mechanical energy, and combinations thereof.

13. The device of claim 12 wherein in situ curing of the flowable mixture forms a drug depot that encapsulates and time releases at least one anti-inflammatory agent and, optionally, any secondary additives.

14. The device of claim 12 wherein a delivery device is used to deliver the flowable mixture to a bone lesion.

15. The device of claim 18 wherein the delivery device is selected from a group comprising a syringe, needle, cannula, or catheter.

16. The device of claim 19 wherein the delivery device has a channel with a cross section not larger than 8 G.

17. The device of claim 1 further comprising at least one biologically active agent.

18. The device of claim 10 wherein the growth factor is BMP-2 or LMP-1 or combinations thereof.

19. The method for treating an osteo-degenerative disease comprising the steps of:
a) the combination of at least one anti-inflammatory agent, and, optionally, at least one secondary additive, with a flowable drug carrier forming a flowable mixture;
b) delivering the flowable mixture to a bone lesion;
c) in situ curing of the mixture forming a rigid drug depot;
d) wherein the drug depot bio-resorbs thereby time releasing the anti-inflammatory agent to treat a degenerative bone disease; and
e) followed by an optional treatment using an osteoinductive material in combination with a growth factor.

20. The method of claim 19 wherein the osteo-degenerative disease is osteolysis caused by an inflammation response to debris particles emitted from a prosthetic implant.

21. The method of claim 19 wherein the energy to activate in situ curing of the flowable mixture is applied during or after delivery of the flowable mixture to a bone lesion.

22. The method of claim 19 wherein the osteo-conductive material comprises a medical grade purified collagen, a biphasic calcium phosphate (BCP), ceramic granules, and growth factors.

23. A surgical kit for treating a bone degenerative disease comprising:
a flowable drug carrier with at least one anti-inflammatory agent and, optionally, at least one secondary additive; a curing agent; a delivery device; a curing device; and written instructions indicating a method of treating an osteo-degenerative disease.
